(19) United States
(12) Patent Application Publication
(10) Pub. No.: US 2008/0214942 A1
(43) Pub. Date: Sep. 4, 2008

(54) APPARATUS AND METHOD FOR MEASURING BLOOD PRESSURE

(75) Inventors: Hyun-Ho OH, Seoul (KR); Bong-Chu Shim, Gyeonggi-Do (KR); Gyoung-Soo Kim, Gyeonggi-Do (KR); Yun-Hee Ku, Gyeongeanqbuk-Do (KR); Seong-Moon Cho, Gyeonggi-Do (KR); Hyung-Ki Hong, Gyeonggi-Do (KR)

Correspondence Address: BIRCH STEWART KOLASCH & BIRCH, PO BOX 747, FALLS CHURCH, VA, 22040-0747, US

(73) Assignee: LG Electronics Inc., Seoul (KR)

(21) Appl. No.: 12/027,913

(22) Filed: Feb. 7, 2008

(30) Foreign Application Priority Data
Feb. 9, 2007 (KR) 10-2007-0013987
Nov. 21, 2007 (KR) 10-2007-0118940

(51) Int. Cl. A61B 5/024; A61B 5/022; A61B 5/103

(52) U.S. Cl. 600/485; 600/500; 600/587

(57) ABSTRACT

Disclosed are an apparatus and method for measuring a blood pressure capable of enhancing accuracy and reliability for a blood pressure. According to the apparatus and method, a blood pressure is obtained by using a pulse transit time (PTT) calculated based on a pulse wave measured with a minimized error, a subject's body information, pulse analysis information, and environment information together measured when measuring the pulse wave.

### RELATED APPLICATION

The present invention relates to subject matter contained in priority Korean Application No. 10-2007-0013987, filed on Feb. 97, 2007 and No. 10-2007-0118940, filed on Nov. 21, 2007, which is herein expressly incorporated by reference in its entirety.

### BACKGROUND OF THE INVENTION

1. Field of the Invention

The present invention relates to an apparatus and method for measuring a blood pressure, and more particularly, to an apparatus and method for measuring a blood pressure capable of enhancing the accuracy and reliability of a blood pressure by minimizing an error.

2. Description of the Conventional Art

A blood pressure refers to the force exerted by circulating blood on the walls of blood vessels.

The blood pressure serves as an important physiological criteria including a lot of information relating to a cardiac output, an elasticity of a blood vessel, and a subject's psychological changes.

The blood pressure includes a systolic blood pressure and a diastolic blood pressure corresponding to a maximum blood pressure and a minimum blood pressure according to systole and diastole, respectively.

A method for measuring a blood pressure is classified into an invasive method and a non-invasive method.

According to the invasive method, catheter is inserted into a blood vessel thus to continuously and precisely measure a blood pressure. However, the invasive method may result in infections and side-effects.

According to the non-invasive method, a cuff is used to detect sound or vibration of a pulse wave by pressurization and decompression, thereby measuring a blood pressure. However, the non-invasive method has a limitation in consecutively measuring a blood pressure. Another non-invasive methods using no cuff include a method for calculating an artery average pressure by analyzing a waveform obtained through a photo plethysmogram (PPG), a method for calculating a blood pressure by a pulse transit time (PTT) calculated through an electrocardiographical (ECG) signal and a photo plethysmograph (PPG) signal, etc.

The invasive and non-invasive methods serve to calculate a blood pressure by physically sensing expansion or contraction of a blood vessel.

The non-invasive method, an indirect measuring method results in some errors. In the case of an optical measuring method, the accuracy of a measured value is influenced by a skin thickness, skin ingredients, a contact degree of a sensor, etc. Furthermore, a diastolic blood pressure has a lower accuracy than a systolic blood pressure in a measuring principle.

In the case of the non-invasive method, an equation of regression commonly applied to all the subjects may not be implemented.

When a pulse wave is measured by a PPG (Photo Plethysmograph) sensor, a pulse wave signal measured from a finger's end may be influenced by a strength of force applied onto the PPG sensor for pressing. That is, a pulse wave signal may be influenced by a pressure of a subject's finger applied onto a PPG sensor for measuring of a pulse wave, and by a pressure of the PPG sensor applied to a subject's finger. Accordingly, a blood pressure calculated based on the pulse wave signal may degrade the reliability.

In order to minimize influence by a pressure applied between the PPG sensor and the subject's finger, methods for analyzing a waveform of a measured pulse wave or compensating the waveform by a pressure sensor have been disclosed. However, the methods require additional complicated processes for analyzing or compensating a waveform of a measured pulse wave, which causes a lot of efforts and time and needs new equipment.

### SUMMARY OF THE INVENTION

Therefore, it is an object of the present invention to provide an apparatus and method for measuring a blood pressure capable of providing a diastolic blood pressure having a high reliability to a subject when measuring a blood pressure, in which a pulse transit time (PTT) is calculated based on a pulse wave and an electrocardiogram measured with a minimized error, a systolic blood pressure is calculated based on the PTT, and a diastolic blood pressure is calculated by the systolic blood pressure, the PTT, pulse analysis information for the measured pulse wave, the subject's body information inputted by the subject, and environment information together measured when measuring the pulse wave.

It is another object of the present invention to provide an apparatus and method for measuring a blood pressure capable of providing an equation of regression that is commonly applied to all the subjects by obtaining a diastolic blood pressure based on the systolic blood pressure, the PTT, pulse analysis information for a measured pulse wave, a subject's body information, and environment information.

It is still another object of the present invention to provide an apparatus and method for measuring a blood pressure capable of simply and precisely calculating a blood pressure by using a PPG sensor without complicated processes requiring much time and efforts, without being influenced by a pressure applied between the PPG sensor and a subject's finger.

To achieve these and other advantages and in accordance with the purpose of the present invention, as embodied and broadly described herein, there is provided an apparatus for measuring a blood pressure, comprising: a sensor unit for measuring at least one of a subject's electrocardiogram and pulse wave; an information input unit for inputting the subjects body information; and a controller for calculating a pulse transit time (PTT) based on the measured electrocardiogram and pulse wave, calculating a first systolic pressure based on the calculated PTT, and calculating a first diastolic pressure based on the calculated first systolic pressure, the PTT, pulse analysis information for the measured pulse wave, the subject's body information inputted by the subject, and environment information together measured when measuring the electrocardiogram or the pulse wave.

To achieve these and other advantages and in accordance with the purpose of the present invention, as embodied and broadly described herein according to a first embodiment, there is provided a method for measuring a blood pressure, comprising: calculating a pulse transit time (PTT) based on a subject's electrocardiogram and pulse wave; calculating a first systolic pressure based on the PTT; and calculating a first diastolic pressure by applying, to an equation of regression, the first systolic pressure, the PTT, pulse analysis information the measured pulse wave, the subject's body information inputted by the subject, and environment information together measured when measuring the electrocardiogram and the pulse wave.

According to a second embodiment of the present invention, there is provided a method for measuring a blood pressure, comprising: pressurizing a subject's finger with pressurizing a bladder, and determining the subject's blood pressure corresponding to a maximum pulse wave among a plurality of measured pulse waves; decompressing the bladder; re-pressurizing the subject's finger to the determined blood pressure thereby measuring a first pulse wave; calculating a pulse transit time (PTT) based on the measured first pulse wave; and calculating a blood pressure based on the calculated PTT.

According to a third embodiment of the present invention, there is provided a method for measuring a blood pressure, comprising: pressurizing a subject's finger with pressurizing a bladder, and determining the subject's blood pressure corresponding to a maximum pulse wave among a plurality of measured pulse waves; decompressing the bladder; re-pressurizing the subject's finger to the determined blood pressure thereby measuring a pulse wave; calculating a pulse transit time (PTT) based on the measured pulse wave; and calculating a blood pressure by applying, to an equation of regression, the PTT the subject's body information inputted by the subject, and environment information together measured when measuring the pulse wave.

According to a fourth embodiment of the present invention, there is provided a method for measuring a blood pressure, comprising: pressurizing a subject's finger with pressurizing a bladder, and determining the subject's blood pressure corresponding to a maximum pulse wave among a plurality of measured pulse waves; decompressing the bladder; re-pressurizing the subject's finger to the determined blood pressure thereby measuring a pulse wave; measuring the subject's electrocardiogram by using an electrocardiogram measuring electrode; calculating a pulse transit time (PTT) based on the measured pulse wave and electrocardiogram; and calculating a blood pressure by applying, to an equation of regression, the PTT, pulse analysis information for the measured pulse wave, and the subject's body information.

According to a fifth embodiment of the present invention, there is provided a method for measuring a blood pressure, comprising: pressurizing a subject's finger with pressurizing a bladder, and determining the subject's blood pressure corresponding to a maximum pulse wave among a plurality of measured pulse waves; decompressing the bladder, re-pressurizing the subject's finger to the determined blood pressure thereby measuring a pulse wave; measuring the subject's electrocardiogram by using an electrocardiogram measuring electrode; calculating a pulse transit time (PTT) based on the measured pulse wave and electrocardiogram; and calculating a blood pressure by applying, to an equation of regression, the PTT, the subject's body information inputted by the subject, and environment information together measured when measuring the pulse wave.

According to a sixth embodiment of the present invention, there is provided a method for measuring a blood pressure, comprising: pressurizing a subject's finger with pressurizing a bladder, and determining the subject's blood pressure corresponding to a maximum pulse wave among a plurality of measured pulse waves; decompressing the bladder; re-pressurizing the subject's finger to the determined blood pressure thereby measuring a pulse wave; measuring the subject's electrocardiogram by using an electrocardiogram measuring electrode; calculating a pulse transit time (PTT) based on the measured pulse wave and electrocardiogram; calculating a first systolic pressure based on the calculated PTT; and calculating a first diastolic pressure by applying, to an equation of regression, the first systolic pressure, the PTT, pulse analysis information for the measured pulse wave, the subject's body information inputted by the subject, and environment information together measured when measuring the electrocardiogram and the pulse wave.

The foregoing and other objects, features, aspects and advantages of the present invention will become more apparent from the following detailed description of the present invention when taken in conjunction with the accompanying drawings.

### BRIEF DESCRIPTION OF THE DRAWING

The accompanying drawings, which are included to provide a further understanding of the invention and are incorporated in and constitute a part of this specification, illustrate embodiments of the invention and together with the description serve to explain the principles of the invention.

In the drawings:

FIG. 1 is a configuration view of an apparatus for measuring a blood pressure according to the present invention;

FIGS. 2A to 2C are sectional views showing a first example of a pressurization means of a sensor unit according to the present invention;

FIGS. 3A and 3B are sectional views showing a first example of a PPG sensor according to the present invention;

FIG. 4 is a perspective view of a non-invasive apparatus for measuring a blood pressure according to a first embodiment of the present invention;

FIG. 5 is a flowchart showing a method for measuring a blood pressure according to a first embodiment of the present invention;

FIG. 6 is a flowchart showing a method for measuring a blood pressure according to a second embodiment of the present invention;

FIGS. 7A and 7B are waveforms for calculating a pulse transit time (PTT) according to the present invention;

FIG. 8 is a waveform for obtaining pulse analysis information according to the present invention;

FIG. 9 is a flowchart showing a method for measuring a blood pressure according to a third embodiment of the present invention;

FIG. 10 is a flowchart showing a method for measuring a blood pressure according to a fourth embodiment of the present invention;

FIGS. 11A and 11B are views for analyzing a diastolic blood pressure (R-sq(adj)) of the present invention and a diastolic blood pressure (R-sq(adj)) of the conventional art; and

FIGS. 12A and 12B are graphs showing a systolic blood pressure and a diastolic blood pressure measured by a non-invasive apparatus for measuring a blood pressure according to the present invention.

### DETAILED DESCRIPTION OF THE PREFERRED EMBODIMENT

Reference will now be made in detail to the preferred embodiments of the present invention, examples of which are illustrated in the accompanying drawings.

Hereinafter, an apparatus and method for measuring a blood pressure according to the present invention will be explained in more detail with reference to the attached drawings.

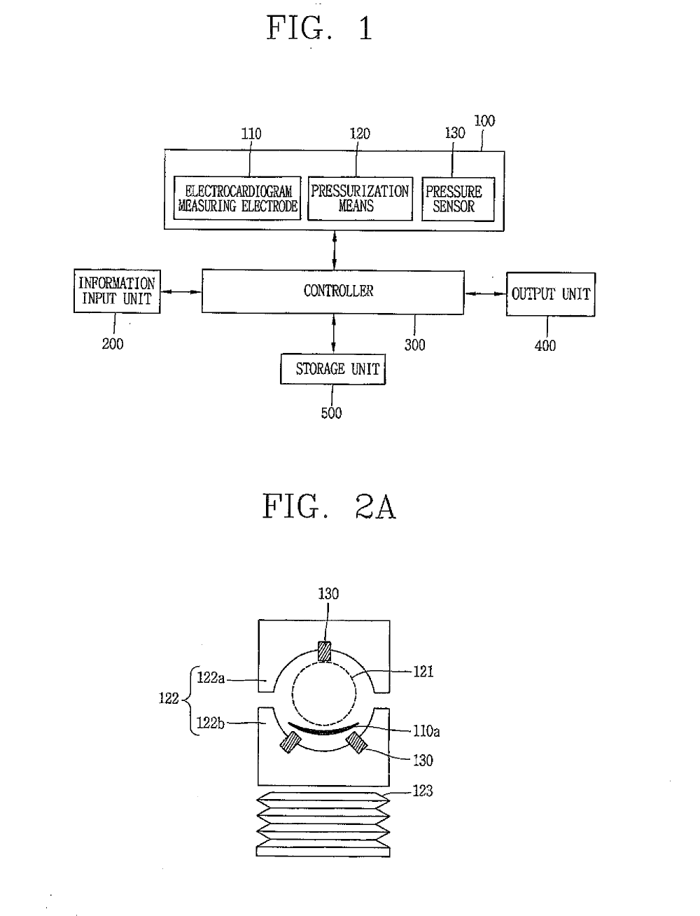

FIG. 1 is a configuration view of an apparatus for measuring a blood pressure according to the present invention.

As shown in FIG. 1, an apparatus for measuring a blood pressure according to the present invention comprises a sensor unit 100 for measuring at least one of a subjects electrocardiogram and pulse wave; an information input unit 200 for inputting the subject's body information; a controller 300 for calculating a pulse transit time (PTT) based on the measured electrocardiogram and pulse wave, calculating a first systolic pressure based on the calculated PTT and calculating a first diastolic pressure based on the calculated first systolic pressure, the PTT, pulse analysis information for the measured pulse wave, the subject's body information inputted by the subject, and environment information together measured when measuring the electrocardiogram or the pulse wave; an output unit 400 for outputting at least one of the first systolic pressure and the first diastolic pressure calculated by the controller 300; and a storage unit 500 for storing at least one of the first systolic pressure and the first diastolic pressure calculated by the controller 300.

The sensor unit 100 includes one or more electrocardiogram measuring electrodes 110 for measuring the subject's electrocardiogram, a pressurization means 120 for pressurizing the subject's finger and releasing the pressurization, and a pressure sensor 130 for measuring the subject's pulse wave.

The electrocardiogram measuring electrode 110, the pressurization means 120, and the pressure sensor 130 of the sensor unit 100 are individually controlled by the controller 300.

An electrocardiogram or pulse wave is measured by using at least one of the electrocardiogram measuring electrode 110, the pressurization means 120, and the pressure sensor 130 of the sensor unit 100.

When measuring the subject's electrocardiogram or pulse wave, the sensor unit 100 also measures at least one of the subject's peripheral temperature, humidity, and air pressure.

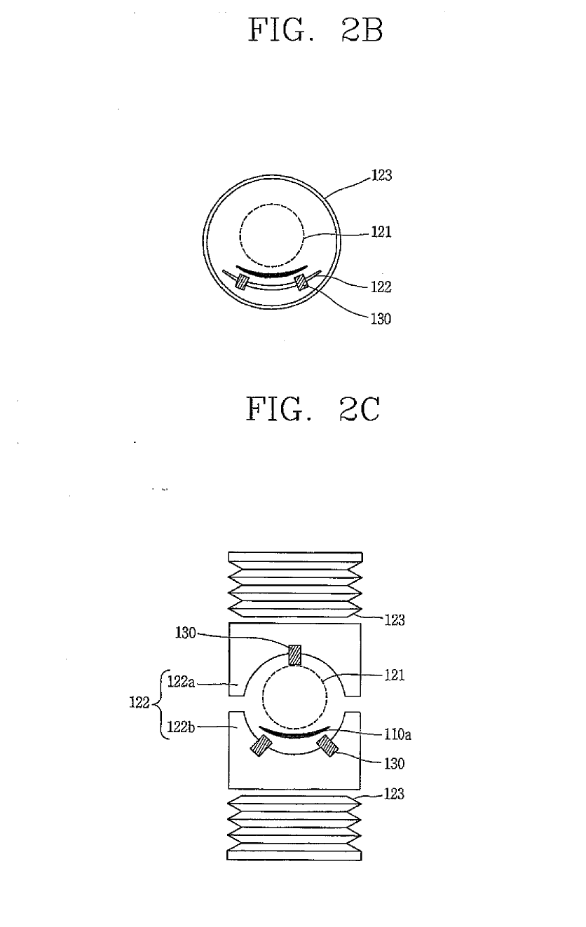

FIGS. 2A to 2C are sectional views showing a first example of a pressurization means of a sensor unit according to the present invention.

As shown in FIG. 2A, the pressurization means 120 includes a supporting portion 122 having a through hole 121 for inserting a subject's finger, and a bladder 123 for pressurizing the through hole 121 and/or the supporting portion 122 so as to pressurize the subject's finger inserted into the through hole 121. The through hole 121 and the bladder 123 serve to facilitate to repeatedly pressurize the subject's finger. As shown in FIGS. 2A to 2C, in order to evenly pressurize an end portion of the subject's finger, the bladder 123 is preferably provided to encompass the through hole 121 (FIG. 2A), or is provided at one or two outer surfaces of the supporting portion 122 (FIGS. 2A and 2C).

The pressure sensor 130 is implemented as a PPG sensor.

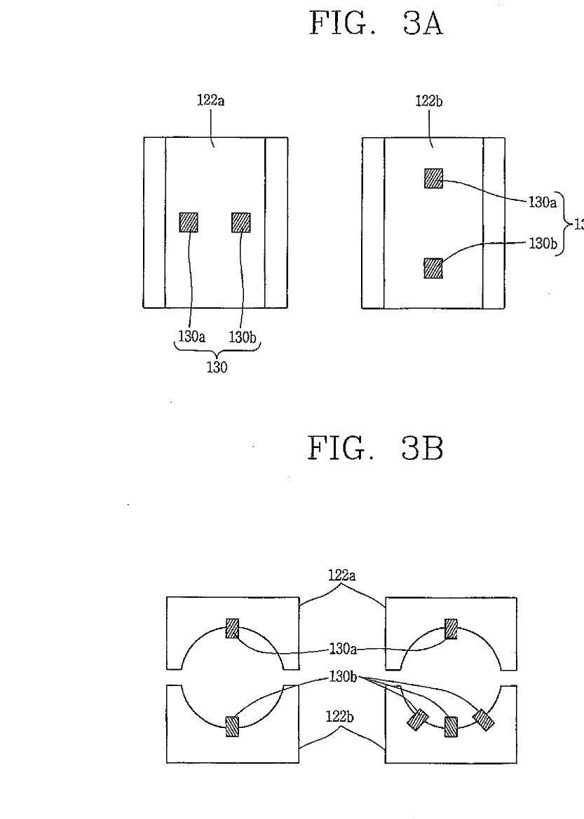

FIGS. 3A and 3B are sectional views showing a first example of a PPG sensor according to the present invention. As shown, the supporting portion 122 is composed of an upper supporting portion 122a and a lower supporting portion 122b, and the PPG sensor 130 is composed of one or more light emitting devices 130a and light receiving devices 130b. The light emitting device 130a and the light receiving device 130b are provided at one or two of the upper supporting portion 122a and the lower supporting portion 122b. More concretely, as shown in FIG. 3A, the PPG sensor 130 may be configured as a reflective type so that the light emitting device 130a and the light receiving device 130b are horizontally or vertically disposed on a lower surface of the subject's finger. As shown in FIG. 3B, the PPG sensor 130 may be configured as a transmissive type so that the light emitting device 130a and the light receiving device 130b are vertically disposed on upper and lower surfaces of the subject's finger.

The information input unit 200 is configured to input the subject's body information including height, weight, age, sex, arm length, etc.

The controller 300 calculates a pulse transit time (PTT) that changes according to an artery pressure by using the subject's electrocardiogram and pulse wave.

Also, the controller 300 calculates a first systolic pressure based on the calculated PTT. Here, the systolic blood pressure is inversely proportional to the square of the PTT.

Also, the controller 300 calculates a diastolic blood pressure by applying, to an equation of regression, the first systolic pressure, the PTT, pulse analysis information for the measured pulse wave, the subject's body information inputted through the information input unit 200, and environment information together measured when measuring the electrocardiogram and the pulse wave.

Also, the controller 300 may determine the subject's blood pressure judged by the pressure sensor 130 to correspond to a maximum pulse wave among a plurality of pulse waves inputted through the pressurization means 120, re-pressurize the subject's finger to the determined blood pressure by controlling the pressurization means 120, and calculate a blood pressure by using pulse wave information obtained at the time of the re-pressurization.

The output unit 400 outputs the first systolic pressure and the first diastolic pressure calculated by the controller 300 to the subject through wire/radio media such as the subject's portable phone, PDA, personal computer, and e-mail.

The storage unit 500 stores the first systolic pressure and the first diastolic pressure calculated by the controller 300, the measured electrocardiogram and pulse wave, the calculated PTT the subject's body information inputted through the information input unit 200, the measured environment information, etc.

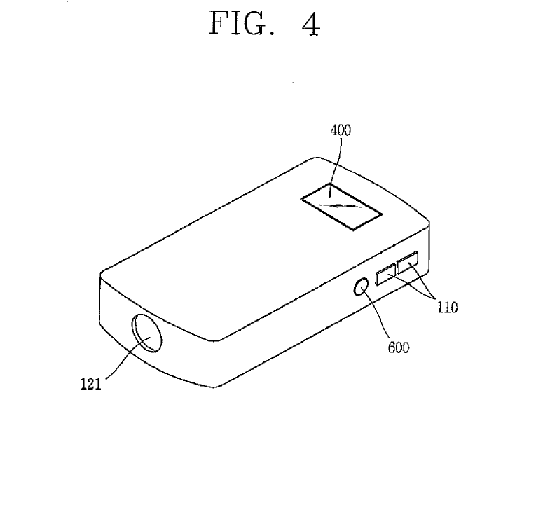

FIG. 4 is a perspective view of a non-invasive apparatus for measuring a blood pressure according to a first embodiment of the present invention.

As shown, the non-invasive apparatus for measuring a blood pressure may be implemented as an independent device to be utilized as a measuring device for exclusive use. In the non-invasive apparatus for measuring a blood pressure, an electrocardiogram measuring electrode 110 for measuring a subjects electrocardiogram is composed of an electrocardiogram measuring inner electrode 110a provided below the thorough hole 121 of the supporting portion 122, and an electrocardiogram measuring outer electrode 110b provided at a side surface of a housing. Under this configuration, a subject's one hand encompasses the non-invasive apparatus with two fingers (e.g., left hand's thumb and index finger) contacting the electrocardiogram measuring outer electrode 110b, whereas the subject's another hand finger (e.g., right hand's index finger) is inserted into the through hole 1212 positioned at a lower end of the apparatus. Then, a switch 600 is turned on/off thus to measure the subject's electrocardiogram and/or pulse wave.

The apparatus may be utilized as an independent one due to its simple configuration and manipulation, or may be mounted in a mobile communication terminal, an MP3 player, a portable video reproducing apparatus, a game machine, etc.

The apparatus is configured to calculate a diastolic blood pressure by using a systolic blood pressure, thereby enhancing the accuracy and reliability of the diastolic blood pressure.

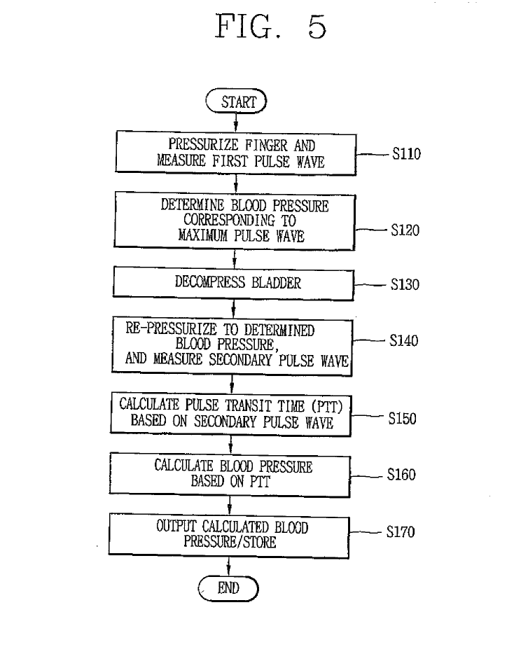

FIG. 5 is a flowchart showing a method for measuring a blood pressure according to a first embodiment of the present invention.

First, a subject's finger is inserted into the pressurization means 120 of the sensor unit 100, and is pressurized by pressurizing the bladder 123 of the pressurization means 120 under control of the controller 300. Then, a pulse wave of the pressurized finger is firstly measured by using a PPG sensor, the pressure sensor 130. Here, a pulse wave of the pressurized finger is secondly measured by using the pressure sensor 130 with a pressure/time period preset by the subject while continuously pressurizing the bladder 123.

The sensor unit 100 may sense whether the subject's finger was inserted into the pressurization means 120 a sensor (not shown) additionally installed at any position of the pressurization means 120, and may control the bladder 123 by the controller 300 based on the sensed result.

As shown in FIG. 2, the subject's finger is easily pressurized by using the bladder 123 containing the air therein. The pressure applied to the subject's finger is also easily released by exhausting the air inside the bladder 123. A strength of a force applied to the subject's finger may be measured by using a pressure sensor 130, or by measuring a torque applied to a motor.

Then, the controller 300 determines the subject's blood pressure corresponding to a maximum pulse wave among a plurality of measured pulse waves. Here, the blood pressure corresponding to a maximum pulse wave and transmitted to the pressure sensor 130 is referred to as a mean blood pressure (MBP). The MBP is expressed by the following formula 1.

(MBP)=1*SBP/3+2*DBP/3 [Formula 1]

Here, the SBP denotes a systolic blood pressure, and the DBP denotes a diastolic blood pressure (S120).

Next, the bladder 123 is decompressed (S130).

Next, the subject's finger is re-pressurized to the MBP thereby to measure a secondary pulse wave (S140).

A pulse transit time (PTT) is calculated based on the measured secondary pulse wave (S150), and a blood pressure is calculated based on the PTT. The process for calculating a blood pressure based on the PTT can be performed by various methods known to those skilled in the art.

As a factor to calculate the blood pressure, the subject's environment information together calculated when measuring the pulse wave may be used. Here, the subject's environment information includes the subject's peripheral temperature, humidity, air pressure, etc. As a factor to calculate the blood pressure, the subject's body information inputted by the subject may be used. Here, the subject's body information includes at least one of height, weight, age, sex, and arm length.

A blood pressure may be calculated by applying the environment information and the body information to an equation of regression.

Blood Pressure=C1*Pulse Transit Time(PTT)+C2*Body information+C3*Environment Information+C4 [Formula 2]

Here, the C1 to C4 are constants obtained through a regression analysis (S160).

Next, the calculated blood pressure may be outputted to the subject through the output unit 400, or may be stored in the storage unit 500 (S170).

As aforementioned, a pulse wave and an MBP become different according to a subject's blood pressure, and the pulse wave becomes different according to a pressure applied to the sensor unit 100 by a subject's finger, or a pressure applied to the subject's finger by the sensor unit 100. That is, as a pressure applied to the subject's finger gradually increases, a pulse wave gradually increases thus to reach a maximum level. When the pressure more increases, the pulse wave decreases. An external pressure corresponding to a maximum pulse wave calculated by the sensor unit 100 is associated with an MBP. In the present invention, a blood pressure corresponding to a maximum pulse wave is predetermined, and a pulse wave is measured within the determined blood pressure. Accordingly, an error resulting from a difference of a pressure applied to the subject's finger is minimized.

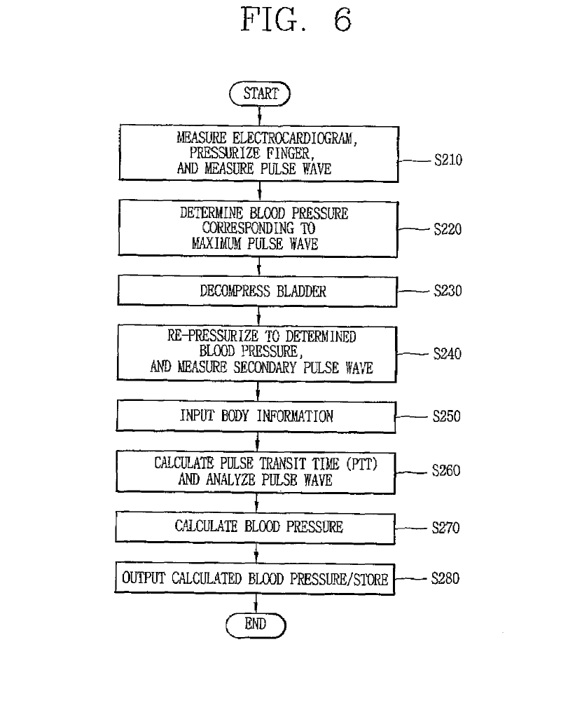

FIG. 6 is a flowchart showing a method for measuring a blood pressure according to a second embodiment of the present invention.

First, a subject's finger is inserted into the pressurization means 120 of the sensor unit 100, and is pressurized by pressurizing the bladder 123 of the pressurization means 120 under control of the controller 300. Then, a pulse wave of the pressurized finger is firstly measured by using a PPG sensor, the pressure sensor 130. Here, a pulse wave of the pressurized finger is secondly measured by using the pressure sensor 130 with a pressure/time period preset by the subject while continuously pressurizing the bladder 123.

The subject's electrocardiogram is measured by using the electrocardiogram measuring electrode 110 of the sensor unit 100 (S210).

Next, the subject's finger is pressurized thus to determine a blood pressure corresponding to a maximum pulse wave (S220).

Next, the bladder 123 is decompressed (S230).

Next, the bladder 123 is re-pressurized to the determined blood pressure thereby to measure a secondary pulse wave (S240).

Steps S220 though S240 are equal to steps S120 through S140 shown in FIG. 5 according to the first embodiment.

Next, the subject's body information is inputted by the information input unit 200 thus to be stored in the storage unit 500.

Here, the subject's body information includes at least one of the subject's height, weight, age, sex, and arm length.

Next, the subject's body mass index (BMI) is calculated by using the subject's height and weight (S250).

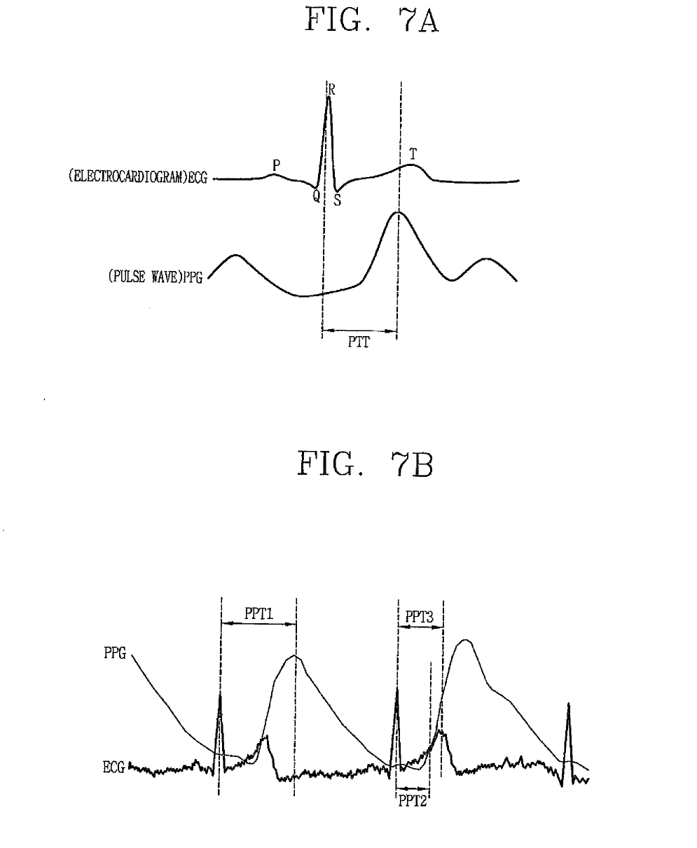

Next, a pulse transit time (PTT) is calculated based on the measured electrocardiogram and secondary pulse wave. The PTT is calculated by measuring a time interval between a point at which an electrocardiographic R wave has a peak value and a point at which an arbitrary value preset by the subject is shown. As shown in FIG. 7A, the PTT is calculated by measuring a time interval between a point at which an electrocardiographic R wave has a peak value and a point at which a pulse wave has a peak value (e.g., a maximum point). As shown in FIG. 7B, the PTT is calculated by measuring a time interval (PTT1, PTT2, PTT3) between a point at which an electrocardiographic R wave has a peak value and a point at which an arbitrary value preset by the subject is shown.

The electrocardiogram waveform is referred to as “PQRST” wave according to the peak position. The PTT is calculated by using the ‘R’ wave, and the ‘R’ wave indicates a time point at which blood is pumped from the heart.

Pulse analysis information is calculated by analyzing a pulse wave measured by the PPG sensor.

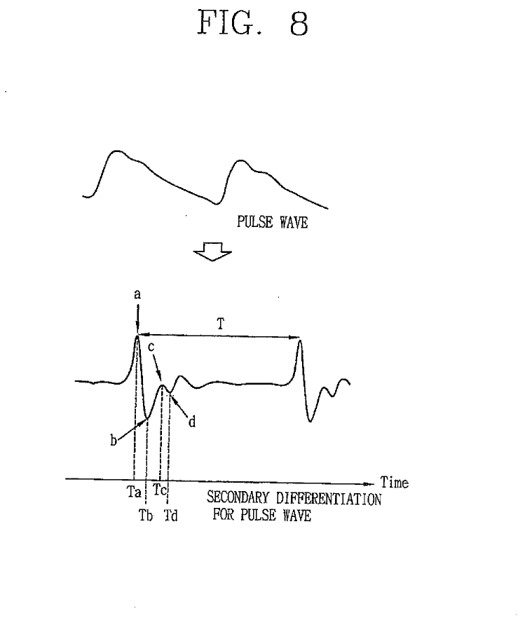

As shown in FIG. 8, the pulse analysis information is calculated by obtaining a secondary differentiated waveform for the measured pulse wave, and includes at least one of each peak value or a blood vessel age (a degree of arterial aging of the subject) of the secondary differentiated waveform for the measured pulse wave. That is, the pulse analysis information includes each peak value (a, b, c and d) of the secondary differentiated waveform for the measured pulse wave, or includes values calculated by combining the peak values (a, b, c and d) to each other by four fundamental rules of arithmetic. For example, a±b, a±c, a±d, b±d, b/a, d/c, (a+b)/c, etc. may be used as the pulse analysis information.

The pulse analysis information includes the number of pulses (60/T) calculated from a time period between two peaks of a firstly differentiated waveform for the pulse wave.

When peak values of a secondary differentiated waveform for the pulse wave are assumed to be sequentially ‘a, b, c and d’, the blood vessel age is calculated by combining the respective peak values each other, which is expressed as the following formula 3.

Blood vessel age(degree of arterial aging)=(−b+c+d)/a [Formula 2]

The higher the blood pressure age is, the better a status of a blood vessel is. The blood vessel age indicating a status of a blood vessel influences on a blood pressure. When thrombus is accumulated on a blood vessel wall, a blood passage in the blood vessel is narrowed thus to increase a resistance of the blood vessel.

The pulse analysis information includes values calculated by combining respective time (Ta, Tb, Tc and Td) corresponding to the respective peak values to each other. For instance, the pulse analysis information includes T1−(Tb−Ta), T2−(Tc−Ta), T3−(Td−Ta), T1′=T1/T, T2′=T2/T, T3′=T3/T, etc. Here, the Ta, Tb, Tc and Td indicate each time corresponding to the ‘a, b, c and d’ of the secondary differentiated waveform for the pulse wave. And, the T denotes a time period of a pulse wave, and 60/T denotes the number of pulses (S260).

Next, the PTT, the pulse analysis information, and the subject's body information are applied to an equation of regression, thereby calculating a blood pressure.

The equation of regression is expressed as the following formula 4, and the blood pressure is calculated by combining the PTT, the pulse analysis information, and the subject's body information to each other.

Blood Pressure=C1*Pulse Transit Time+C2*Pulse Analysis Information+C3*Body information+C4 [Formula 4]

Here, the C1 to C4 are constants obtained through a regression analysis.

As a factor to calculate the blood pressure, the subject's environment information together calculated when measuring the subject's electrocardiogram and pulse wave may be used. Here, the subject's environment information includes temperature, humidity, air pressure, and the like (S270).

Next, the calculated blood pressure may be outputted to the subject through the output unit 400, or may be stored in the storage unit 500 (S280).

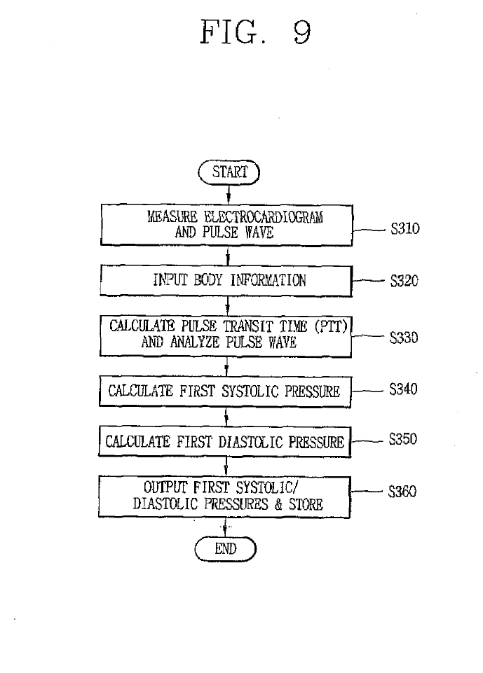

FIG. 9 is a flowchart showing a method for measuring a blood pressure according to a third embodiment of the present invention.

First, a subject's electrocardiogram and pulse wave are measured by using a sensor unit 100.

When measuring the subject's electrocardiogram and pulse wave, the subject's environment information including at least one of temperature, humidity, and air pressure is also measured (S310).

Next, the subject's body information is inputted through the information input unit 200 (S320).

Next, a pulse transit time (PTT) is calculated based on the measured electrocardiogram and pulse wave, and pulse wave is analyzed (S330). Steps S320 and S330 are equal to steps S250 and S260 shown in FIG. 6 according to the second embodiment.

Next, a first systolic pressure is calculated based on the calculated PTT (S340). Here, the systolic blood pressure is inversely proportional to the square of the PTT.

Next, a first diastolic pressure is calculated based on the first systolic pressure, the PTT, pulse analysis information for the measured pulse wave, and the subject's body information inputted by the subject.

The first diastolic pressure is calculated by combining the first systolic pressure, the PTT, pulse analysis information for the measured pulse wave, the subject's body information, and the environment information to each other, and is expressed as the following formula 5.

First diastolic pressure=C1*Pulse Transit Time(PTT)+C2*Pulse Analysis Information+C3*Body Information+C4*Environment Information+C5*First systolic pressure+C6 [Formula 5]

Here, the C1 to C6 are constants obtained through a regression analysis, and other factors rather than the above factor may be used to calculate the first diastolic pressure (S350).

Next, at least one of the calculated first systolic and diastolic pressures is outputted to the subject through the output unit 400, or is stored in the storage unit 500 (S360).

The method for measuring a blood pressure according to the present invention comprises calculating a pulse transit time (PTT) based on a subject's electrocardiogram and pulse wave; calculating a first systolic pressure based on the PTT; and calculating a diastolic blood pressure based on the first systolic pressure, the PTT, pulse analysis information for the measured pulse wave, the subject's body information inputted by the subject, and environment information together measured when measuring the electrocardiogram and the pulse wave. Accordingly, one object of the present invention is to provide an equation of regression commonly applied to all the subjects when measuring a blood pressure.

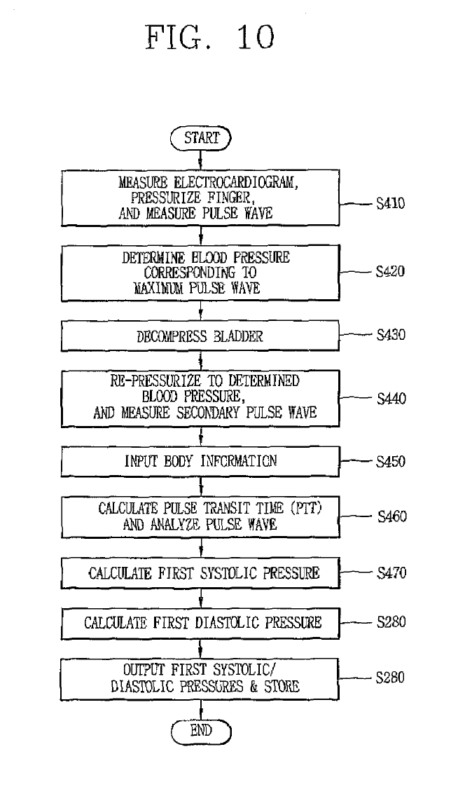

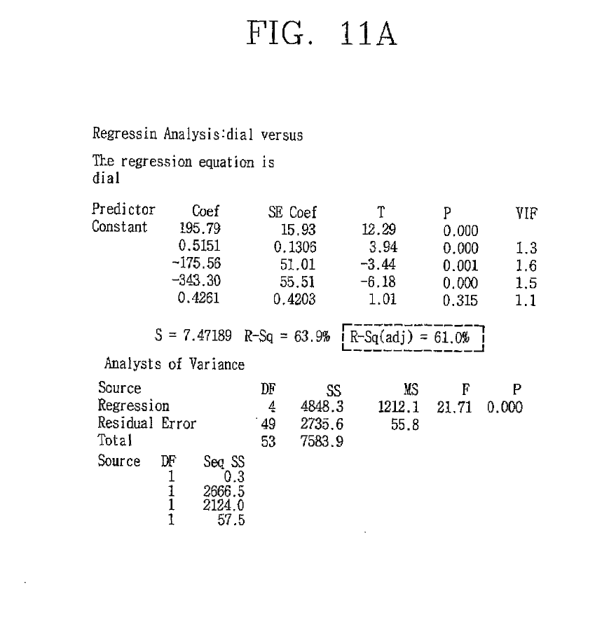

FIG. 10 is a flowchart showing a method for measuring a blood pressure according to a fourth embodiment of the present invention.

The method comprises measuring a subject's electrocardiogram and pulse wave (S410); pressurizing the subject's finger with pressurizing a bladder 123 thereby determining the subject's blood pressure corresponding to a maximum pulse wave (S420); decompressing the bladder 123 (S430); re-pressurizing the subject's finger to the determined blood pressure thereby measuring a secondary pulse wave (S440); inputting the subject's body information through an information input unit 200 (S450); and calculating a pulse transit time (PTT) based on the measured electrocardiogram and secondary pulse wave, and analyzing the measured pulse wave (S460). Steps S410 though S460 are equal to steps S210 through S260 shown in FIG. 6 according to the second embodiment.

In step S420 for determining the subject's blood pressure corresponding to a maximum pulse wave, a second systolic blood pressure and a second diastolic blood pressure applied to the formula 1 are obtained by using the measured pulse wave.

Next, a first systolic pressure is obtained based on the calculated PTT (S470).

Next, a first diastolic pressure is obtained based on the first systolic pressure, the PTT, pulse analysis information for the measured pulse wave, and the subject's body information inputted by the subject (S480). S470 and S480 are equal to steps S340 and S350 shown in FIG. 9 according to the third embodiment.

Next, at least one of the calculated first systolic and diastolic pressures is outputted to the subject through the output unit 400, or is stored in the storage unit 500 (S490).

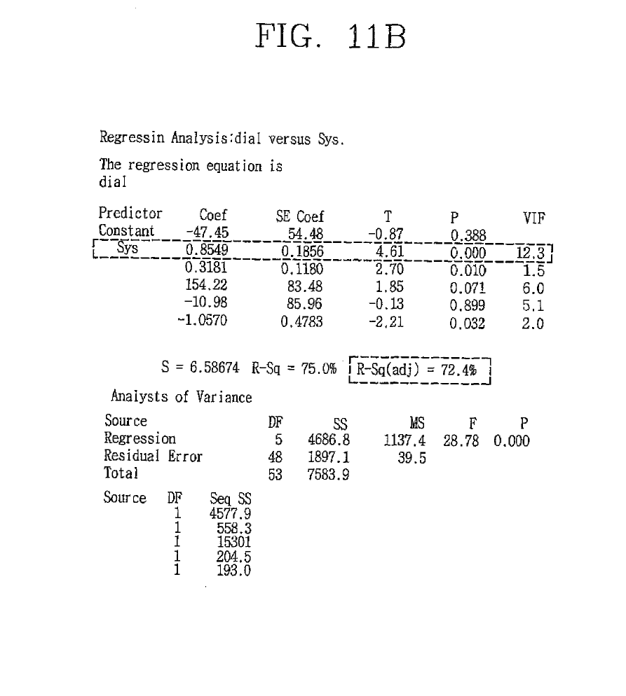

Hereinafter, a diastolic blood pressure (R-sq(adj)) obtained by using a systolic blood pressure according to the present invention will be compared with a diastolic blood pressure (R-sq(adj)) of the conventional art with reference to FIG. 11.

FIGS. 11A and 11B are views for analyzing a diastolic blood pressure (R-sq(adj)) of the present invention and a diastolic blood pressure (R-sq(adj)) of the conventional art.

As shown in FIG. 11A, the conventional diastolic blood pressure (R-sq(adj)) is 61.0%.

On the contrary, as shown in FIG. 11B, the diastolic blood pressure (R-sq(adj)) of the present invention is 72.4%, which is more accurate than the conventional one.

The analysis is performed by the controller 400 by using a mini-tab software, a regression type calculation program stored in the storage unit 500. Here, the R denotes a correlation coefficient, and the pressure (R-sq(adj)) denotes a coefficient of determination for an equation of regression applied to a general regression analysis. The higher the (R-sq(adj)) is, the higher the reliability is.

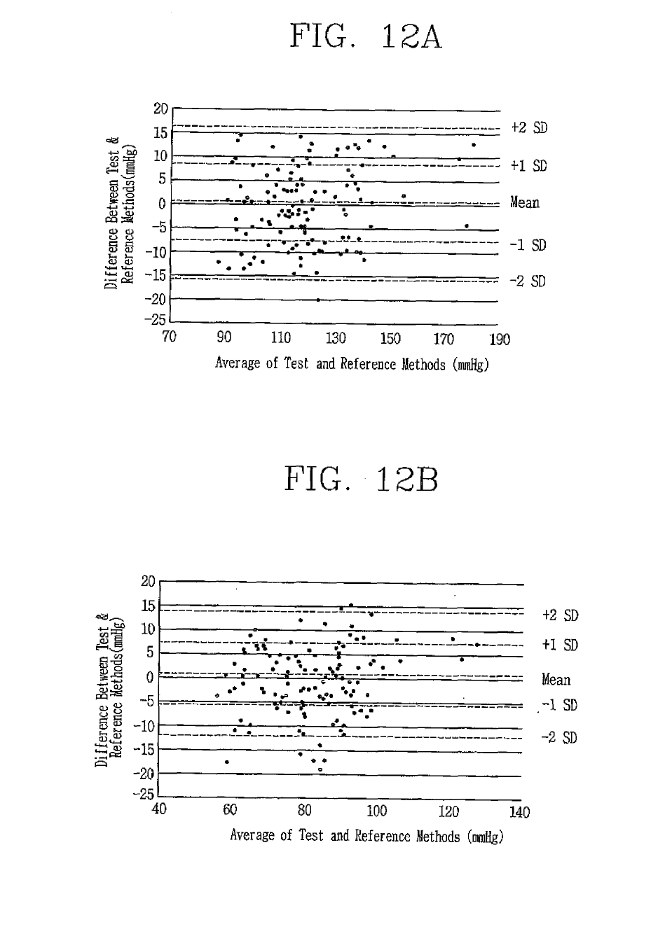

Hereinafter, with reference to FIG. 12, will be explained Bland-Altman plot that shows the conventional systolic and diastolic blood pressures measured by using a mercury blood pressure measuring apparatus and systolic and diastolic blood pressures of the present invention measured by using a non-invasive blood pressure apparatus.

A Bland-Altman plot is a method of data plotting used in comparing two different assays or tests.

FIGS. 12A and 12B are graphs comparing a blood pressure measured by a non-invasive apparatus according to the present invention with a reference blood pressure measured by the conventional stethoscopic method using a mercury blood pressure measuring apparatus in a Bland Altman plot manner. According to specifications relating to certification of a blood pressure measuring apparatus (SP10 and EN1060), a blood pressure measuring apparatus has to satisfy accuracy of ‘mean mean error+/−5 mmHg, SD 8 mmHG. The apparatus for measuring a blood pressure according to the present invention shows a systolic blood pressure of mean error −0.2 mmHg, SD 8.0 mmHG as shown in FIG. 12A, and shows a diastolic blood pressure of mean error −0.5 mmHg, SD 7.1 mmHg as shown in FIG. 12B. Accordingly, it is judged that the apparatus for measuring a blood pressure according to the present invention satisfies accuracy required in specifications for a blood pressure measuring apparatus.

As aforementioned, in the apparatus and method for measuring a blood pressure according to the present invention, a pulse transit time (PTT) is calculated based on a subject's electrocardiogram and pulse wave having a minimized error; a systolic blood pressure is calculated based on the PTT; and a diastolic blood pressure is calculated based on the systolic blood pressure, the PTT, pulse analysis information for the measured pulse wave, the subject's body information inputted by the subject, and environment information together measured when measuring the electrocardiogram and the pulse wave. Accordingly, a diastolic blood pressure having a higher reliability can be provided to the subject when measuring a blood pressure.

Furthermore, in the apparatus and method for measuring a blood pressure according to the present invention, a diastolic blood pressure is calculated based on the systolic blood pressure, the PTT, pulse analysis information for the measured pulse wave, the subject's body information, and the environment information. Accordingly, an equation of regression that is commonly applied to all the subjects at the time of measuring a blood pressure can be provided.

Moreover, in the apparatus and method for measuring a blood pressure according to the present invention, a blood pressure can be easily, simply and precisely calculated by using a PPG sensor without performing complicated processes requiring much time and efforts, without being influenced by a pressure applied between the PPG sensor and a subject's finger.

The foregoing embodiments and advantages are merely exemplary and are not to be construed as limiting the present invention. The present teachings can be readily applied to other types of apparatuses. This description is intended to be illustrative, and not to limit the scope of the claims. Many alternatives, modifications, and variations will be apparent to those skilled in the art. The features, structures, methods, and other characteristics of the exemplary embodiments described herein may be combined in various ways to obtain additional and/or alternative exemplary embodiments.

As the present features may be embodied in several forms without departing from the characteristics thereof, it should also be understood that the above-described embodiments are not limited by any of the details of the foregoing description, unless otherwise specified, but rather should be construed broadly within its scope as defined in the appended claims, and therefore all changes and modifications that fall within the metes and bounds of the claims, or equivalents of such metes and bounds are therefore intended to be embraced by the appended claims.

What is claimed is:

1. A method for measuring a blood pressure, comprising:
calculating a pulse transit time (PTT) based on a subjects electrocardiogram and pulse wave;
calculating a first systolic pressure based on the PTT, and
calculating a first diastolic pressure by applying, to an equation of regression, the first systolic pressure, the PTT, pulse analysis information for the measured pulse wave, the subject's body information inputted by the subject, and environment information together measured when measuring the electrocardiogram and the pulse wave.

2. The method of claim 1, further comprising outputting or storing the calculated first systolic and/or diastolic blood pressure.

3. The method of claim 1, wherein the step of measuring a pulse wave comprises:
pressurizing a subject's finger with pressurizing a bladder, and determining the subject's blood pressure corresponding to a maximum pulse wave among a plurality of measured pulse waves;
decompressing the bladder; and
re-pressurizing the subjects finger to the determined blood pressure thereby measuring a pulse wave.

4. The method of claim 3, wherein the subject's blood pressure corresponding to a maximum pulse wave is obtained by a following formula,
Mean Blood Pressure(MBP)=1*SBP/3+2*DBP/3,
wherein the SBP (Systolic Blood Pressure) denotes a second systolic blood pressure, and the DBP (Diastolic Blood Pressure) denotes a second diastolic blood pressure.

5. The method of claim 3, wherein in the step of decompressing the bladder, air inside the bladder is exhausted out.

6. The method of claim 1, wherein the PTT corresponds to a time interval between a point at which an electrocardiographic R wave has a peak value and a point at which a preset pulse wave is shown.

7. The method of claim 1, wherein the first systolic pressure is is inversely proportional to the square of the PTT.

8. The method of claim 1, wherein the pulse analysis information comprises at least one of peak values of secondary differentiated waveform for the measured pulse wave.

9. The method of claim 8, wherein the pulse analysis information comprises a blood vessel age.

10. The method of claim 9, wherein the blood vessel age is obtained by a following formula 2,
Blood vessel age(degree of arterial aging)=(−b+c+d)/a,
wherein the a, b, c and d indicate constants preset so as to correspond first to fourth peak values of a secondary differentiated waveform for the measured pulse wave.

11. The method of claim 1, wherein the subject's body information comprises at least one of the subject's height, weight, age, sex, and arm length.

12. The method of claim 1, wherein the environment information comprises at least one of the subjects peripheral temperature, humidity, and air pressure together measured when measuring the subject's pulse wave or electrocardiogram.

13. The method of claim 1, wherein an equation of regression of the first diastolic pressure is obtained by a following formula 3,
First diastolic pressure=C1*Pulse Transit Time(PTT)+C2*Pulse Analysis Information+C3*Body Information+C4*Environment Information+C5*First systolic pressure+C6,
wherein the C1 to C4 are constants obtained through a regression analysis.

14. A method for measuring a blood pressure, comprising:
pressurizing a subject's finger with pressurizing a bladder, and determining the subject's blood pressure corresponding to a maximum pulse wave among a plurality of measured pulse waves;
decompressing the bladder;
re-pressurizing the subject's finger to the determined blood pressure thereby measuring a first pulse wave;
calculating a pulse transit time (PTT) based on the measured first pulse wave; and
calculating a blood pressure based on the calculated PTT.

15. A method for measuring a blood pressure, comprising:
pressurizing a subject's finger with pressurizing a bladder, and determining the subject's blood pressure corresponding to a maximum pulse wave among a plurality of measured pulse waves;
decompressing the bladder;
re-pressurizing the subject's finger to the determined blood pressure thereby measuring a pulse wave;
calculating a pulse transit time (PTT) based on the measured pulse wave; and
calculating a blood pressure by applying, to an equation of regression, the PTT, the subject's body information inputted by the subject, and environment information together measured when measuring the pulse wave.

16. The method of claim 15, wherein the equation of regression is obtained by a following formula 4,
Blood Pressure=C1*Pulse Transit Time(PTT)+C2*Body information+C3*Environment Information+C4,
wherein the C1 to C4 are constants obtained through a regression analysis.

17. A method for measuring a blood pressure, comprising:
pressurizing a subject's finger with pressurizing a bladder, and determining the subject's blood pressure corresponding to a maximum pulse wave among a plurality of measured pulse waves;
decompressing the bladder;
re-pressurizing the subject's finger to the determined blood pressure thereby measuring a pulse wave;
measuring the subject's electrocardiogram by using an electrocardiogram measuring electrode;
calculating a pulse transit time (PTT) based on the measured pulse wave and electrocardiogram; and
calculating a blood pressure by applying, to an equation of regression, the PTT, analysis information for the pulse wave, and the subject's body information.

18. The method of claim 17, wherein the equation of regression is obtained by a following formula 5,
Blood Pressure=C1*Pulse Transit Time+C2*Pulse Analysis Information+C3*Body information+C4,
wherein, the C1 to C4 are constants obtained through a regression analysis.

19. A method for measuring a blood pressure, comprising:
pressurizing a subject's finger with pressurizing a bladder, and determining the subject's blood pressure corresponding to a maximum pulse wave among a plurality of measured pulse waves;
decompressing the bladder;
re-pressurizing the subject's finger to the determined blood pressure thereby measuring a pulse wave;
measuring the subject's electrocardiogram by using an electrocardiogram measuring electrode;
calculating a pulse transit time (PTT) based on the measured pulse wave and electrocardiogram; and
calculating a blood pressure by applying, to an equation of regression, the PTT the subject's body information inputted by the subject, and environment information together measured when measuring the pulse wave.

20. The method of claim 19, wherein the equation of regression is obtained by a following formula 6,
Blood Pressure=C1*Pulse Transit Time(PTT)+C2*Pulse Analysis Information+C3*Body Information+C4*Environment Information+C5,
wherein the C1 to C5 are constants obtained through a regression analysis.

21. A method for measuring a blood pressure, comprising:
pressurizing a subject's finger with pressurizing a bladder, and determining the subject's blood pressure corresponding to a maximum pulse wave among a plurality of measured pulse waves;
decompressing the bladder;
re-pressurizing the subject's finger to the determined blood pressure thereby measuring a pulse wave;
measuring the subject's electrocardiogram by using an electrocardiogram measuring electrode;
calculating a pulse transit time (PTT) based on the measured pulse wave and electrocardiogram;
calculating a first systolic pressure based on the calculated PTT; and
calculating a first diastolic pressure by applying, to an equation of regression, the first systolic pressure, the PTT, pulse analysis information for the measured pulse wave, the subject's body information inputted by the subject, and environment information together measured when measuring the electrocardiogram and the pulse wave.

22. The method of claim 21, wherein an equation of regression of the first diastolic pressure is obtained by a following formula 7,
First diastolic pressure=C1*Pulse Transit Time(PET)+C2*Pulse Analysis Information+C3*Body Information+C4*Environment Information+C5*First systolic pressure+C6,
wherein the C1 to C6 are constants obtained through a regression analysis.

23. An apparatus for measuring a blood pressure, comprising:
a sensor unit for measuring at least one of a subject's electrocardiogram and pulse wave;
an information input unit for inputting the subject's body information; and
a controller for calculating a pulse transit time (PTT) based on the measured electrocardiogram and pulse wave, calculating a first systolic pressure based on the calculated PTT, and calculating a first diastolic pressure based on the calculated first systolic pressure, the PTT, pulse analysis information for the measured pulse wave, the subject's body information inputted by the subject, and environment information together measured when measuring the electrocardiogram or the pulse wave.

24. The apparatus of claim 23, further comprising:
an output unit for outputting at least one of the first systolic pressure and the first diastolic pressure calculated by the controller; and
a storage unit for storing at least one of the first systolic pressure and the first diastolic pressure calculated by the controller.

25. The apparatus of claim 23, wherein the sensor unit comprises:
one or more electrocardiogram measuring electrodes for measuring the subject's electrocardiogram;
a pressurization means for pressurizing the subject's finger and releasing is the pressurization; and
a pressure sensor for measuring the subject's pulse wave.

26. The apparatus of claim 25, wherein the pressure sensor is a PPG (Photo Plethysmograph) sensor.

27. The apparatus of claim 26, wherein the pressurization means comprises:
a supporting portion having a through hole; and
a bladder for pressurizing the through hole and/or the supporting portion.

28. The apparatus of claim 27, wherein the supporting portion is composed of an upper supporting portion and a lower supporting portion, and the PPG sensor is composed of one or more light emitting devices and light receiving devices.

29. The apparatus of claim 28, wherein the light emitting device and the light receiving device are provided at one or two of the upper supporting portion and the lower supporting portion.

30. The apparatus of claim 27, wherein the bladder is provided to encompass the through hole 121, or is provided at one or two outer surfaces of the supporting portion.

31. The apparatus of claim 23, wherein the controller utilizes the pulse analysis information including at least one of each peak value or a blood vessel age of a secondary differentiated waveform for the measured pulse wave.

32. The apparatus of claim 23, wherein the controller utilizes the subject's body information including at least one of the subject's height, weight, age, sex, and arm length.

33. The apparatus of claim 23, wherein the controller utilizes the environment information including at least one of the subject's peripheral temperature, humidity, and air pressure together measured when measuring the subject's pulse wave or electrocardiogram.

34. The apparatus of claim 23, wherein the controller calculates the first diastolic pressure by a following formula,
First diastolic pressure=C1*Pulse Transit Time(PTT)+C2*Pulse Analysis Information+C3*Body Information+C4*Environment Information+C5*First systolic pressure+C6.
